(19) United States
(12) Patent Application Publication
(10) Pub. No.: US 2026/0265099 A1
(43) Pub. Date: Sep. 10, 2026

(54) FLUID RESERVOIR

(71) Applicant: David Jessop Harker, New Castle, UT (US)

(72) Inventor: David Jessop Harker, New Castle, UT (US)

(21) Appl. No.: 19/074,347

(22) Filed: Mar. 8, 2025

(51) Int. Cl. C02F 3/28; C12M 1/00; C12M 1/107

(52) U.S. Cl. CPC C02F 3/2866; C12M 23/18; C12M 23/36; C12M 27/20; C02F 2203/006

(57) ABSTRACT

A fluid reservoir system can include a fluid reservoir and a mixer. The mixer can include multiple mixer inlets positioned around an upper perimeter of the fluid reservoir. A conduit structure can extend from the mixer inlets to one or more mixer outlets below the mixer inlets. A pump system can be fluidly connected to the conduit structure. The mixer can be structured to use the pump system to recirculate fluid in the reservoir by pumping fluid from at or near the top the reservoir through different subsets of the mixer inlets at different times, through the conduit structure, and through the one or more mixer outlets at or near the bottom back into the fluid reservoir. Also, a sediment removal system can use head pressure to flow sediment from the bottom of the fluid reservoir to a sediment removal reservoir.

### BACKGROUND

Fluid reservoirs are used to hold biological fluid including biological material such as urine and feces from domestic animals, food waste, and/or other biological material. Covers have been placed on such reservoirs to facilitate collection of biogases emitted from the fluid in the reservoirs. Such biogases can be used as a renewable source of fuel.

### SUMMARY

Whatever the advantages of previous fluid reservoir systems and their uses, they have neither recognized the aspects described and claimed herein, nor the advantages produced by such aspects.

According to one aspect, a technique can include mixing fluid, which can include biological material, in a fluid reservoir during a first period of time with a first mixer system configuration. The mixing can include passing fluid from the fluid reservoir through a first subset of one or more mixer inlets including less than all of a set of mixer inlets positioned around the fluid reservoir. The mixing can also include passing fluid through a mixer conduit structure from the first subset of one or more mixer inlets and back into the fluid reservoir through at least one of one or more mixer outlets below the set of mixer inlets. Also, the technique can include mixing fluid in the fluid reservoir during a second period of time that is different from the first period of time with a second mixer system configuration that is different from the first mixer system configuration. The technique may include reconfiguring from the first mixer system configuration to the second mixer system configuration, and this reconfiguring may include a controller sending signals to one or more system components and/or performing manual actions. The mixing can include passing fluid from the fluid reservoir through a second subset of one or more mixer inlets including less than all of the set of mixer inlets, with the second subset of one or more mixer inlets being different from the first subset of one or more mixer inlets. The mixing can also include passing fluid through the mixer conduit structure from the second subset of one or more mixer inlets and back into the fluid reservoir through at least one of the one or more mixer outlets below the set of mixer inlets.

According to another aspect, a fluid reservoir system can include a fluid reservoir and a mixer. The mixer can include multiple mixer inlets positioned around an upper perimeter of the fluid reservoir. A conduit structure can extend from the mixer inlets to one or more mixer outlets below the mixer inlets. A pump system can be fluidly connected to the conduit structure. The mixer can be structured to use the pump system to recirculate fluid to the reservoir by pumping fluid from the top of the reservoir through different subsets of the mixer inlets at different times, through the conduit structure, and through the one or more mixer outlets back into the fluid reservoir.

According to another aspect, a technique can include pumping fluid from a sediment removal reservoir. The pumping can lower a fluid surface of the sediment removal reservoir to a level that is at a lower altitude than a level of a fluid surface of a fluid reservoir that extends at least partially into ground so that there is a difference in altitude of the fluid surface of the fluid reservoir and the fluid surface of the sediment removal reservoir. The technique can further include clearing accumulated sediment from a bottom of the fluid reservoir by flowing the sediment in fluid from the fluid reservoir to the sediment removal reservoir through a conduit structure using a pressure head due to the difference in altitude of the fluid surface of the fluid reservoir and the fluid surface of the sediment removal reservoir.

According to yet another aspect, stored fluid comprising biological material in a fluid reservoir can be mixed. The mixing can include passing recirculating fluid from the fluid reservoir through one or more mixer inlets at a top of the stored fluid in the fluid reservoir, and recirculating the recirculating fluid through a mixer conduit structure from the one or more mixer inlets and back into the fluid reservoir through at least one mixer outlet at a bottom of the stored fluid in the fluid reservoir.

This Summary is provided to introduce a selection of concepts in a simplified form. The concepts are further described below in the Detailed Description. This Summary is not intended to identify key features or essential features of the claimed subject matter, nor is it intended to be used to limit the scope of the claimed subject matter. Similarly, the invention is not limited to implementations that address the particular techniques, tools, environments, disadvantages, or advantages discussed in the Background, the Detailed Description, or the attached drawings.

### BRIEF DESCRIPTION OF THE DRAWINGS

FIG. 1 is a front-left-top perspective view of features of a fluid reservoir system.

FIG. 2 is an enlarged view cut out along line 2 of FIG. 1.

FIG. 3 is an enlarged view cut out along line 3 of FIG. 1.

FIG. 4 is a front-loft-bottom perspective view of the features of the fluid reservoir system of FIG. 1.

FIG. 5 is an enlarged view cut out along line 5 of FIG. 4.

FIG. 6 is an enlarged view cut out along line 6 of FIG. 4.

FIG. 7 is a front view of the features of the fluid reservoir system of FIG. 1.

FIG. 8 is a right-side view of the features of the fluid reservoir system of FIG. 1.

FIG. 9 is a top view of the features of the fluid reservoir system of FIG. 1.

FIG. 10 is a bottom view of the features of the fluid reservoir system of FIG. 1.

FIG. 11 is a schematic view of features of a fluid reservoir system, with features like the features in the fluid reservoir system of FIG. 1 having the same labels. The features of the fluid reservoir system of FIG. 11 are not illustrated to scale or accurately positioned in FIG. 11. Also, FIG. 11 illustrates a system with multiple inlets and multiple outlets along its bottom, such as a narrow elongate bottom.

FIG. 12 is a perspective view of mixer inlet valve in an open position.

FIG. 13 is a side view of the mixer inlet valve of FIG. 12.

FIG. 14 is a section view taken along line 14 -14 of FIG. 13.

FIG. 15 is a perspective view of the mixer inlet valve of FIG. 12 in a closed position.

FIG. 16 is a side view of the mixer inlet valve of FIG. 15.

FIG. 17 is a section view taken along line 17 -17 of FIG. 16.

FIG. 18 is a front-left-top perspective view of features of another fluid reservoir system.

FIG. 19 is an enlarged view cut out along line 13 of FIG. 18.

FIG. 20 is a front-loft-bottom perspective view of the features of the fluid reservoir system of FIG. 18.

FIG. 21 is an enlarged view cut out along line 15 of FIG. 20.

FIG. 22 is a block diagram of a suitable computing environment in which one or more of the described aspects may be implemented.

FIG. 23 is a flowchart illustrating a fluid reservoir technique.

FIG. 24 is a flowchart illustrating another fluid reservoir technique.

FIG. 25 is a flowchart illustrating yet another fluid reservoir technique.

The description and drawings may refer to the same or similar features in different drawings with the same reference numbers.

### DETAILED DESCRIPTION

### I. General Discussion

Examples of features of fluid reservoir systems are discussed below. It has been found that prior fluid reservoir systems such as digesters may have one or more problems. Problems may include an inability to efficiently remove the sediment in a covered flat bottom containment which results in shortened service life, and reduced capacity as the sediment accumulates. Also, problems may include down time, large expenses, and/or liner and cover damage in the clean out process. Also, problems may also include floated and failing liners as a result of trapped gas in near flat bottom designs. This may be reduced or eliminated with enough slope to the top. Problems may also include difficulty reducing heat loss. (The heat loss may be proportion to the surface area of the liner and of the cover.) In most configurations, a deeper steeper sloped containment (i.e., a deeper steeper sloped fluid reservoir) can have less surface area per gallon stored. Problems may also include difficulty obtaining higher gas yields. It has been found that higher gas yields may be achieved using improved mixing system design, and removal of unproductive sediment. Problems may also include difficulty reducing cover maintenance costs. It has been found that a smaller cover can result from a deeper design, thus a smaller top cover per gallon of containment and reduction of or elimination of cover penetrations stress points.

It has been found that with regards to the product, liquid with organic solids and the fine dirt or sand that is commonly with it can have a higher specific gravity than water. This can result in heavier particles slowly falling through the water toward the bottom where they form sediment at the bottom if not kept in liquid suspense, and the lighter fluid and floating particles may tend to rise toward and accumulate at the top.

It has been found that reservoir systems such as those discussed herein can be designed to utilize the understanding discussed above to obtain an improved mixture such as a full mix or a more full mix with less mixing energy. This design may include a steeply sloped bottom to a sediment removal drain sump and to the mixing injection location. The design can use a piping circulation circuit to collect the lighter liquid and floating particles from mixing inlet ports that are installed close to the top of the liquid, and with the use of a high volume low pressure pump, this lighter fluid collected at the top can be pumped to one or more injection points at the bottom. This injecting of the lighter low solids liquid directly into the bottom (toward where the heavier liquid and particles can be falling) is to infuse the accumulating higher solids product with the low solids liquid to keep the solids in liquid suspense and to provide an updraft of fluid in a counter flow direction of what the solids may be slowly falling toward to maintain a more homogeneous product. For applications with high solids product that tend to have floating particles (such as that tend to form a crust at the top), the design can use multiple (such as eight in an example illustrated in the figures) individually valved skimmers installed around the top to remove the floating crust forming particles with the top lighter liquid, and to enable controlling the mixing fluid flow path from the bottom central injection point to the selected open skimmer or skimmers. The valves on the skimmers can be opened in turn (in other words there may be up to eight or more zones depending on the applied configuration). This may provide focused liquid mixing to each one of the zones of the containment in turn. In other words, all of the zones can have a turn of having focused mixing in that zone to provide a design with more of a full mix in a large containment. The sediment that slides down the sloped bottom to the sediment evacuation sump may be removed from the bottom with the use of a pipe going to an external containment (such as a sediment removal reservoir), utilizing a difference of the liquid level, which can create head pressure to cause the transfer.

The fluid reservoir systems may include a digester system that produces biogas, which is given off from biological material (matter that comes from a living or once-living organism, such as manure, food waste, and/or biological material grown or purchased for use in producing biogas) in the fluid being held in the fluid reservoir. The fluid can include liquids and/or mixtures of liquids with solids and/or semisolids, such as where solids or semisolids are suspended in liquids. The reservoir may be an in-ground lagoon, where at least a portion of the fluid reservoir extends down into the ground. However, the reservoir may be some other type of container or tank, such as a fully or partially above-ground tank. For example, a reservoir may include a lower portion with a sloped bottom (which may be at least partially in ground) and an upper portion with vertical walls, such as a tank with vertical walls. However, features discussed herein may apply to other types of fluid reservoirs, including above-ground tank reservoirs and non-digesting fluid reservoirs.

Solids such as manure solids and the fine dirt or sand that are commonly with the manure can have a higher specific gravity than water. This can result in the solids slowly falling through the water and forming sediment at the bottom. In a fluid reservoir, the solids and fluid may be removed after they have produced the available biogas from them. For example, after years of use, digesters or other fluid reservoirs may accumulate sediment at the bottom of the reservoir, so that they eventually need to be shut down and emptied to have the sediment cleaned out of them. These cleaning procedures commonly require removing the covers from the fluid reservoirs, which can be costly, especially if the covers get damaged.

Some of these cleaning procedures can involve draining and cleaning out the bottom of tank digesters. At least some features of the fluid reservoir systems discussed herein may assist in dealing with solids that tend to build up in the bottoms of fluid reservoirs. As will be discussed more below, the fluid reservoir systems can include sloped sides that extend down to a smaller bottom, such as a single small bottom point or area, or a narrow elongate bottom area.

In some examples, pressure from the fluid in the fluid reservoir can force the solids out of a drain port or sediment removal inlet such as a pipe at a bottom of the fluid reservoir, to drain the solids from the bottom of the reservoir. Some solids may still build up in the bottom, but those solids may be limited to small solid mounds around one or more drain parts. Normally the ports can be closed, but they can be opened periodically to drain out the solids or sediment from the bottom of the fluid reservoir.

In the full mix design a mixer pump is used to provide mixing action and to help keep solids suspended in the fluid of the fluid reservoir. For example, the mixer pump can pump fluid between high inlets at or near the top, such as the skimmers discussed below, which may skim solid material such as crust from the top surface of the fluid in the reservoir. Fluid can be pumped from the upper inlets to one or more lower outlets, which can be at or near the bottom of the fluid reservoir. Thus, fluid can be pumped and recirculated from the top of the fluid reservoir through the upper inlets, through a conduit structure, to the lower outlets, and back into the fluid reservoir. The size and positions of the inlets may be designed according to the needs of a particular system, but enough inlets can be included to get sufficiently uniform mixing, with the fluid rising from the bottom to the top of the fluid reservoir. This upward flow from the bottom pipe to the top pipes may help to keep the solids suspended in the fluid, and to inhibit settling of those solids to the bottom of the fluid reservoir and to provide a full mix design. As with other features discussed herein, the drain port feature and the mixer can be used alone or in combination with each other.

As an alternative example of the inlets, the inlets may include perforations in the pipes in place of the individually valved skimmers. The perforations can be spaced along a pipe that extends around a periphery of the fluid reservoir.

In an example, the fluid reservoir may be a covered fluid reservoir biological material digester, where the fluid reservoir includes a sloped bottom such as an inverted pyramid shape (as illustrated in the figures) with a sloped bottom leading to a small drain area; or a V cone design that can be in the shape of an inverted cone with a sloped bottom leading to a small drain area; or a alternatively a linear V-shaped reservoir, with sloped sides leading to an elongate narrow drain area (such as a channel or service track). The fluid reservoir may include vertical walls extending above the sloped bottom, such as in a tank reservoir. The vertical walls and/or the sloped walls may be fully in the ground, partially in the ground, or fully above ground in different designs. For example, the sloped bottom may be sloped at an angle between about 15 degrees to about 60 degrees, relative to horizontal (but an angle lower that 15 degrees or more than 60 degrees could be used), such as about 35 degrees or a 1:3 slope (one unit vertical to three units horizontal), or a 2:3 slope (two units vertical to three units horizontal), or some other slope such as a 1:1 slope. The bottom drain area can be narrow compared to the width of the reservoir, such as less than 25% of the width of the reservoir or less than 10% of the width of the reservoir. The bottom drain area may be narrow in one direction such as with a narrow long bottom drain area, or narrow in all directions such as with a drain area that is small in length and width. As an example, the sloped sides may slope down to a single drain point or a narrow bottom that may be just wide enough to get machinery into it, such as about eight to ten feet wide, or possibly narrower. This steep V-shaped design can have advantages in that solids or sediments may slide down the sloped sides to the focused mixer outlet and/or drain zone. It may also have other advantages, such as inhibiting biogas that may form under the geomembrane from lifting and floating the membrane, as can happen in low slope manure ponds. Other advantages of such a design may include higher holding capacity per amount of liner material; a smaller top cover per amount of fluid in containment; reduced heating costs because of less heat loss, because this design has less surface area per amount of fluid in containment, compared to other in ground lagoons or covered lagoon digesters; and less odor and less evaporation per gallon stored because of much less top fluid surface exposed per gallon of containment in a lagoon application. However, as noted below, the claims herein should not be construed as being limited to such advantages or others discussed herein unless such advantages are stated in the claims.

As discussed above, the fluid reservoir system can include a covered fluid reservoir biological material digester (such as a biowaste digester) that can include drain ports at the bottom of the fluid reservoir, where the drain ports are typically closed but can be opened to drain solid materials from the bottom of the reservoir. The drain ports can lead to drainpipes that drain solid materials to a sump pit due to force from the head pressure from the fluid in the fluid reservoir, with a fluid surface in the sump pit being lower than a fluid surface of the fluid reservoir.

Also, as discussed above, the fluid reservoir can include a covered fluid reservoir biological material digester that can use a mixer pump to pump fluid from higher points in the fluid reservoir to lower points in the reservoir, to produce an upward flow in the fluid reservoir, thereby tending to keep solids suspended in the fluid held in the fluid reservoir. The mixer can include at least one outlet at or near a bottom of the fluid reservoir and at least one inlet at or near the top of the fluid reservoir (at least partially submerged in the fluid, but near the top surface of the fluid in the fluid reservoir), and a pump that pumps the fluid between the top inlets and the bottom outlets. Also, the mixer can be structured to use a pump system to recirculate fluid by pumping fluid from the top of the reservoir through different subsets of the mixer inlets at different times (which can create focused flow in different zones or areas of the fluid reservoir at different times), through the conduit structure, and through one or more mixer outlets back into the reservoir.

In a manure digester, floating partials may accumulate and form a floating crust that can be a problem if not resolved. As will be discussed more below, a fluid reservoir system can include controlled skimmers that may help to remove solid material such as crust that may accumulate at a surface of the fluid in the reservoir. In an example, one or more skimmers can operate as inlets for the mixer and can each have an automatically or manually operated valve such as a gate valve that can be operated in turn to enable controlling the flow path from the bottom central injection port to each one of the skimmers in turn, possibly as part of subsets of multiple skimmers. This may provide controlled and directed mixing to all sectors of the reservoir. Also, this may be done without moving parts inside the reservoir containment (the area enclosed by the liner and cover of the reservoir). For example, the valves for the skimmers, other valves in the system, and/or pumps in the system can be located outside the reservoir containment. Thus, the valves of the skimmers, other valves in the system, and/or pumps for the system can be fully serviceable externally with the digester full and operational.

When the mixer pump is running for the mixer, the top lighter fluid and crust forming floating solids can flow into the open skimmers. The fluid flowing through the skimmers can then flow through a pipe into the pump where the crust may be broken up in the process. That fluid can then be reinjected into the central bottom where the heavier high solids may be falling. This may infuse the high solids fluid with the low solids fluid and provide an updraft to help keep the solids in suspense and obtain more of a full mix design.

The system may include at least two pipes to remove the post digest product, including a normal top overflow, and a sediment removal pipe. It is expected that during normal operation a digester operator may adjust the frequency of the discharge from the bottom of a digester to remove the least productive product, to maximize gas production and reduce odor production. In other words, it is expected that the digester manager will, through some testing, learn how often or what percentage of the fluid to discharge from the bottom for most productive use of a particular biological material digester.

### II. First Example Fluid Reservoir System

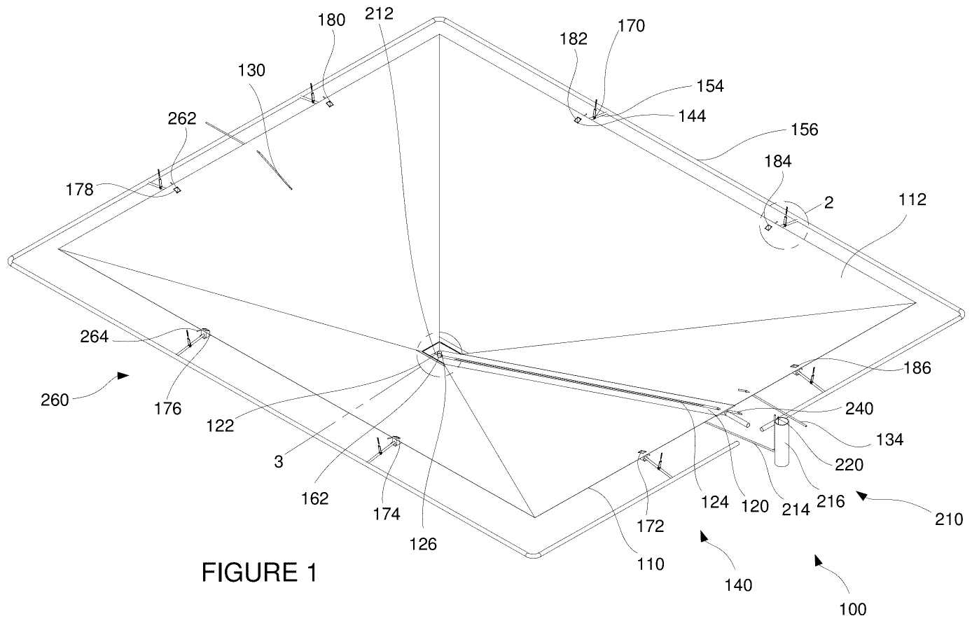

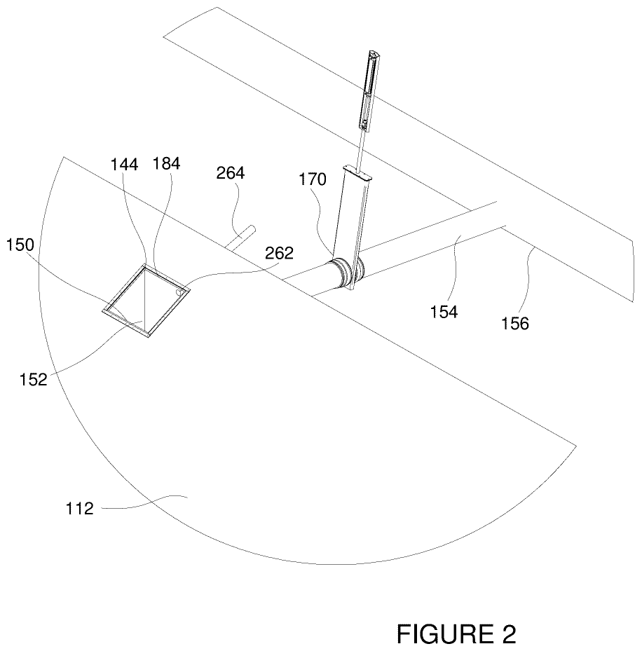

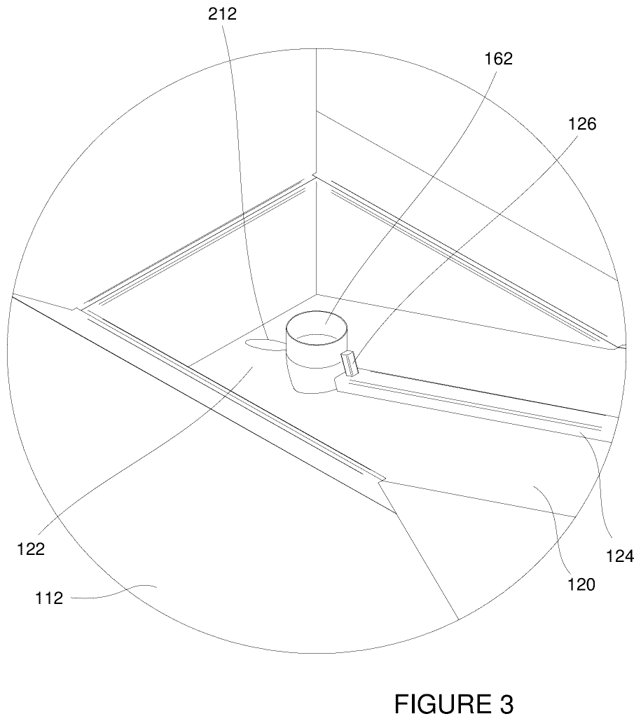

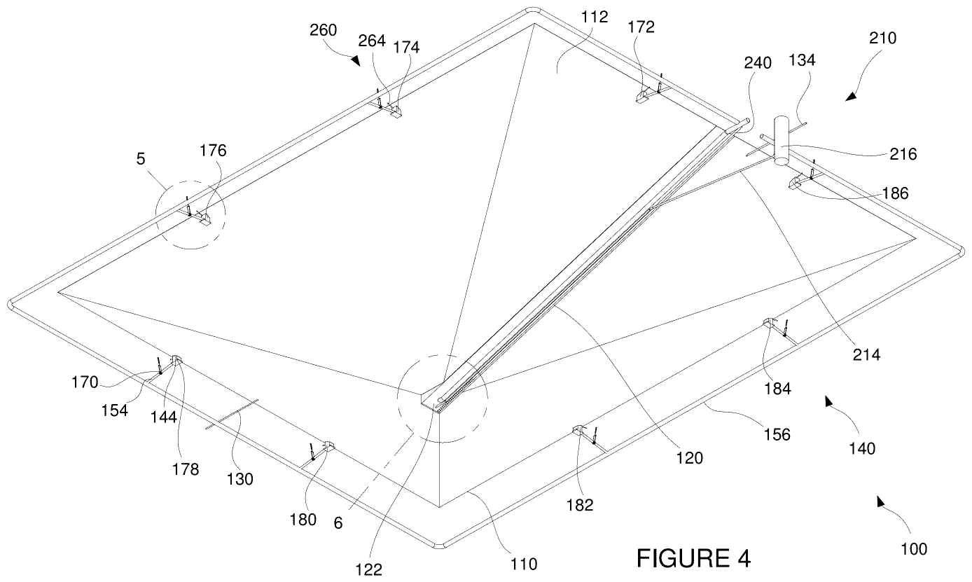

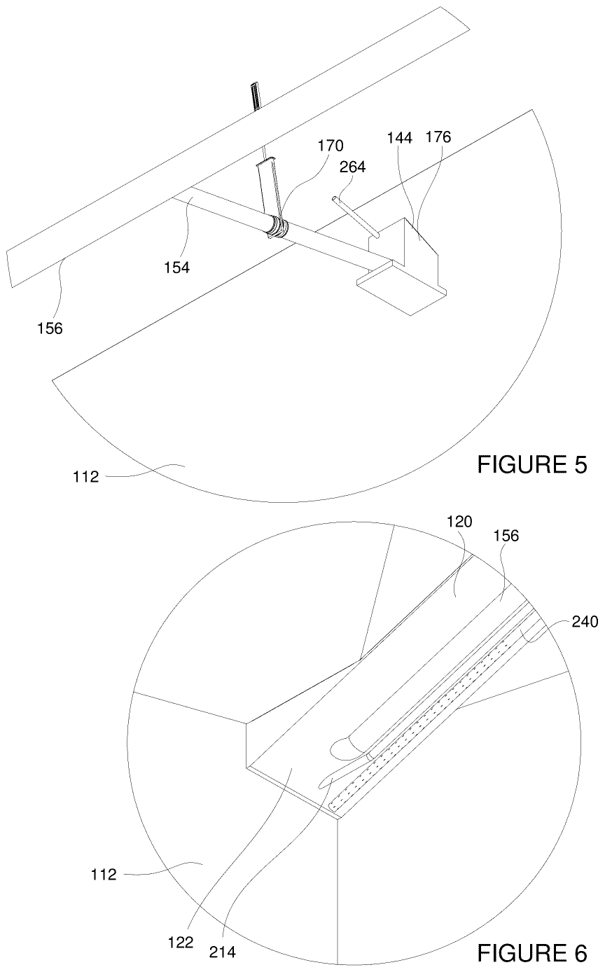

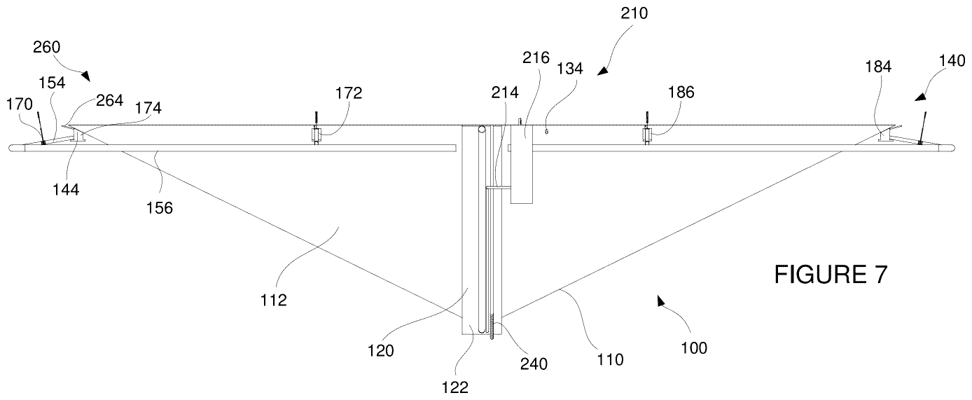

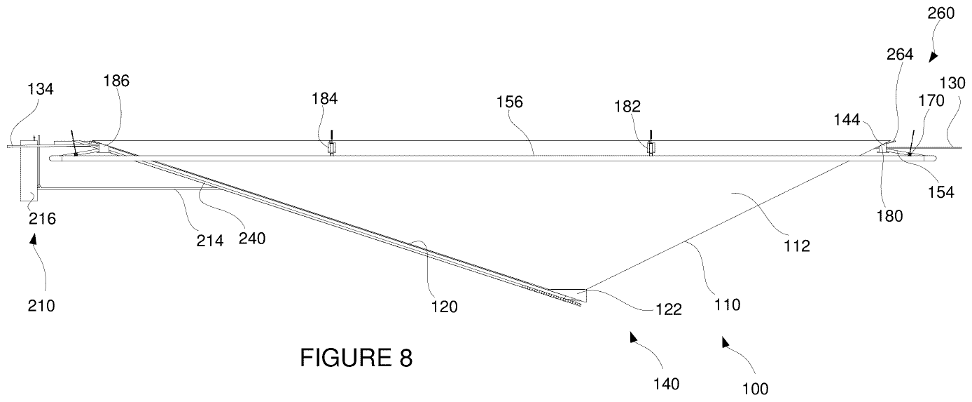

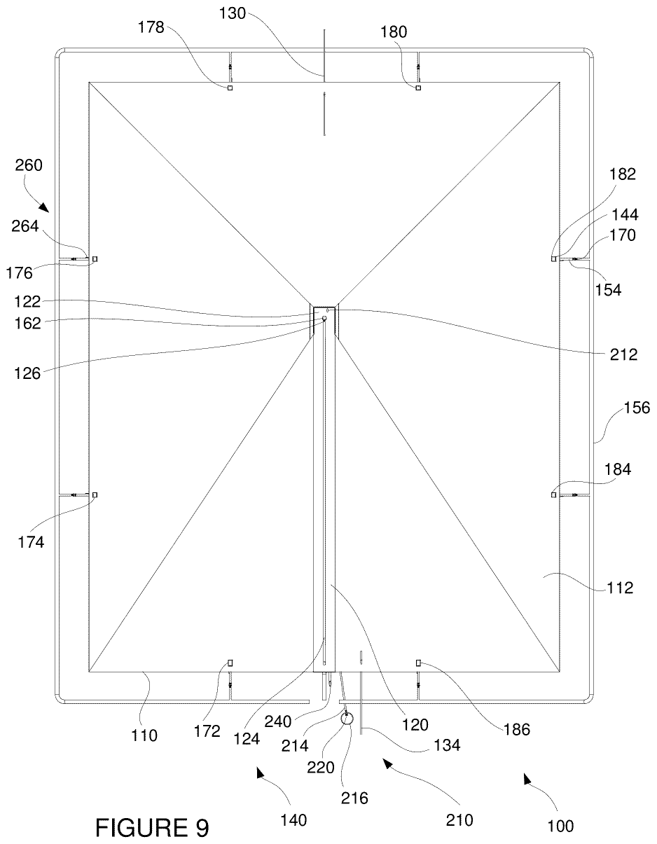

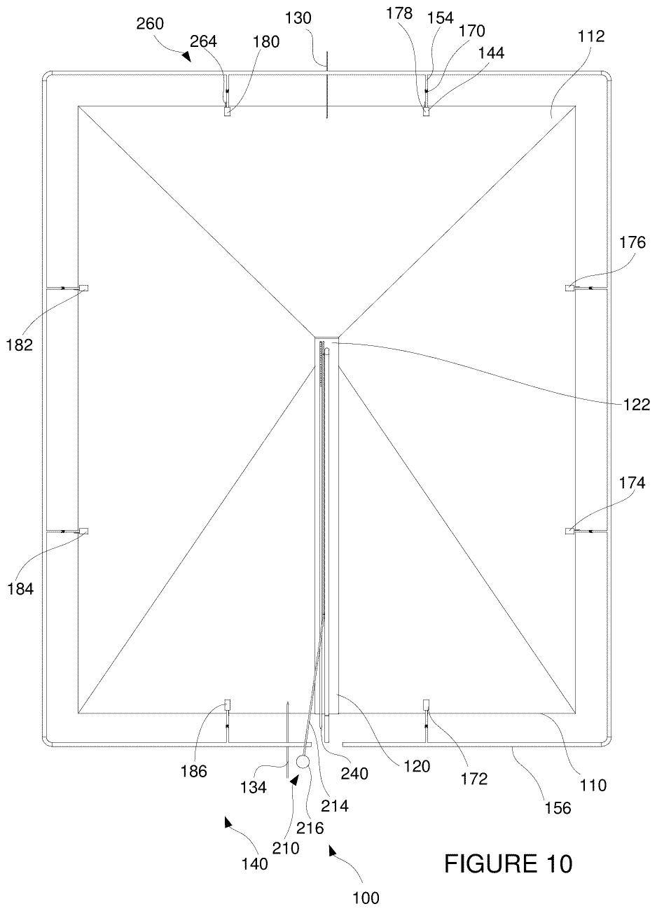

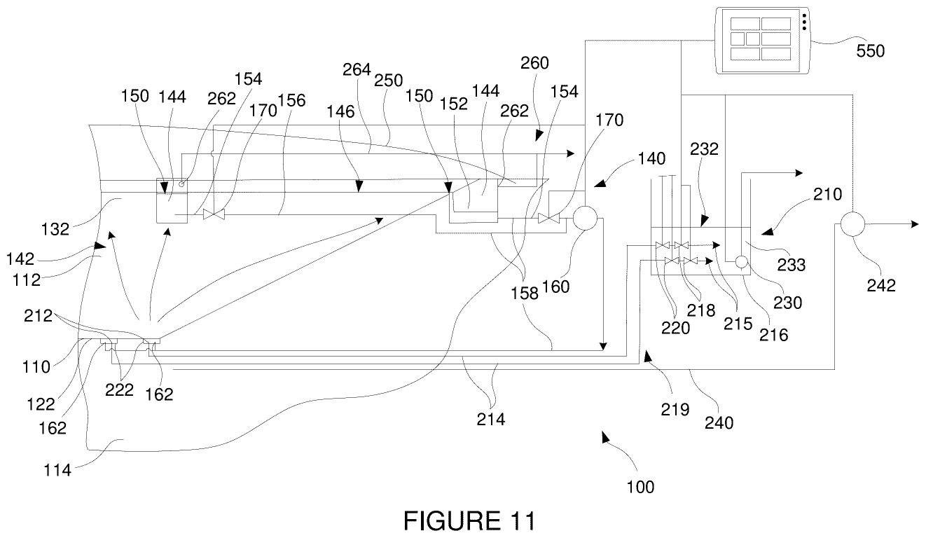

Referring to FIG. 1-10 and the schematic diagram of FIG. 11, a fluid reservoir system 100 will be discussed. The schematic diagram of FIG. 11 includes schematic representations of features of the fluid reservoir system 100 illustrated in FIGS. 1-10, but FIG. 11 includes schematic representations of additional features not illustrated in FIGS. 1-10, such as valves, pumps, and a controller for the reservoir system. Also, FIG. 11 represents multiple mixer outlets and sediment removal inlets along an elongate narrow bottom area (such as where the fluid reservoir forms a V-shape with an elongate bottom), rather than a single mixer outlet and a single sediment removal inlet in a single small bottom area as illustrated in the illustrations of FIGS. 1-10.

The fluid reservoir system 100 can include a fluid reservoir 110, which can include a liner 112, which can extend into the ground 114 (illustrated schematically in FIG. 11) and be supported by the ground 114, with the liner 112 generally forming an inverted pyramid shape. The pyramid shape could include rounded edges between its four sides instead of the sharp edges illustrated in the figures. Indeed, an inverted cone shape can be used instead of the inverted pyramid shape. As an example, the liner 112 may be a geo membrane. The fluid reservoir 110 can also include a service ramp 120, which can be a concrete track extending from an upper periphery of the fluid reservoir 110 down into the fluid reservoir 110, to a bottom drain area 122. The service ramp 120 and the bottom drain area 122 can be formed of concrete. Also, the liner 112 can be secured to the service ramp 120 and to the bottom drain area 122 in a watertight manner, such as with edges of the liner 112 being clamped to the concrete of the service ramp 120 and the bottom drain area 122.

The service ramp 120 can also include a guide rail 124 extending up from the service ramp 120 and extending along the service ramp 120. The service ramp 120 can also include a stop 126 extending up from the service ramp 120 at or near the bottom drain area 122. Vehicles may be driven up and down the service ramp 120 during construction of the fluid reservoir 110 and/or at other times when the fluid reservoir has been drained. Also, a submersible pump (not shown) can be lowered down the service ramp 120 toward the bottom drain area 122 (such as with the use of a winch) to drain the fluid reservoir 110. Also, for a reservoir that has an elongate bottom drain area, the concrete track of the service ramp can extend down to the bottom drain area and along the bottom drain area.

The fluid reservoir system 100 can also include a fluid inlet 130 (and possibly multiple fluid inlets) through which fluid can enter the stored fluid 132 (see FIG. 11) in the fluid reservoir 110. The fluid reservoir 110 can also include an overflow outlet 134 (and possibly multiple overflow outlets) through which fluid can overflow and exit the fluid reservoir 110. The fluid from the overflow outlet 134 can flow to another reservoir, such as a post digestate storage if the fluid reservoir system 100 is a biological material digester.

The fluid reservoir system 100 can also include a mixer 140. The mixer 140 can be structured to create an upward flow 142 (see schematic illustration in FIG. 11) of stored fluid 132 in the fluid reservoir 110. The mixer 140 can include upper mixer inlets 144. Each mixer inlet 144 can be a skimmer, which can skim material from a top surface 146 of the stored fluid 132 of the fluid reservoir 110, such as solid fluid that may be part of a crust that has built up on the top surface 146 of the stored fluid 132 of the fluid reservoir 110. For example, fluid can flow over an edge 150 of a mixer inlet 144 from the stored fluid 132 and into a holding chamber 152 of the mixer inlet 144. An inlet conduit 154 of a conduit structure 156 can lead from a bottom area of the holding chamber 152 of each mixer inlet 144 so that recirculating fluid 158 (see FIG. 11) can flow from the holding chamber 152 out into the conduit structure 156. The liner 112 can be sealed to the mixer inlets 144 such as by being clamped to edges around each mixer inlet 144, such as using bars that are fastened to the mixer inlet 144 around a hole in the liner 112, with the liner 112 being clamped between the bars and concrete material of the mixer inlet 144.

The conduit structure 156 can include one or more pipes fluidly connected to the inlet conduits 154. The conduit structure 156 can extend to a mixer pump 160 and from the mixer pump 160 to one or more mixer outlets 162 at the bottom drain area 122 of the fluid reservoir 110. Thus, recirculating fluid 158 can flow into the mixer inlets 144 from the stored fluid 132, through the conduit structure to the mixer pump 160, from the mixer pump 160 to the mixer outlet 162, and from the mixer outlet 162 back into the stored fluid 132 bottom of the fluid reservoir 110. This can create the flow 142 of stored fluid 132 from the mixer outlet 162 through the stored fluid 132 in the fluid reservoir 110 and up to the mixer inlets 144, which can be positioned above the mixer outlet.

Each inlet conduit 154 can include a mixer inlet valve 170 (such as an air cylinder actuated gate valve), so that each valve can be selectively opened or closed to allow or disallow fluid to flow through the corresponding mixer inlet 144 and into the conduit structure 156. A subset of the mixer inlet valves 170 can be opened at a time so that fluid only flows through an active subset of all the mixer inlets 144 that are positioned around the periphery of the fluid reservoir 110, and the active subset can change over time by opening some of the mixer inlet valves 170 and closing others of the mixer inlet valves 170. This can allow the fluid flow of the mixer 140 to be focused on different areas or zones of the stored fluid 132 in the fluid reservoir 110 at different times, such as focusing the flow in one area of the fluid in the fluid reservoir 110 (between the mixer outlets 162 and the currently active subset of the mixer inlets 144 for the first period of time) during a first period of time and in a different second area of the fluid in the fluid reservoir 110 (between the mixer outlets 162 and the currently active subset of the mixer inlets 144 for the second period of time) during a second period of time. This mixing can proceed to control the fluid flow to multiple general areas and possibly to all general areas of the fluid reservoir 110 in turn. As an example, referring to FIG. 1, for the sake of illustration, the eight mixer inlets 144 of the fluid reservoir system 100 can be numbered sequentially in a clockwise direction as viewed from above the fluid reservoir 110. Thus, the reservoir system 100 can include a first mixer inlet 172, a second mixer inlet 174, a third mixer inlet 176, a fourth mixer inlet 178, a fifth mixer inlet 180, a sixth mixer inlet 182, a seventh mixer inlet 184, and an eighth mixer inlet 186. However, the fluid reservoir system 100 can have different numbers of mixer inlets in locations that are different from those illustrated in the figures, and those mixer inlets may be structured differently from those illustrated in the figures.

In an example, during a first period of time, the first mixer inlet 172, the second mixer inlet 174, and the sixth mixer inlet 182 can be active (i.e., their mixer inlet valves 170 can be open) while fluid is being pumped from the mixer inlets in that first subset and to the mixer outlet 162, creating an upward flow of stored fluid 132 in the fluid reservoir 110 from the mixer outlet 162 to the mixer inlets in that first subset.

Then, the active mixer inlet subset can be switched from the first subset to a second subset. For example, some of the mixer inlet valves 170 can be opened and other mixer inlet valves 170 can be closed, so that during a second period of time, mixer inlet valves 170 can be open only for the second mixer inlet 174, the third mixer inlet 176, and the seventh mixer inlet 184, with those three mixer inlets being the second subset. Thus, during the second period of time, the fluid can be pumped from the second subset of mixer inlets to the mixer outlet 162, creating an upward flow of stored fluid 132 in the fluid reservoir 110 from the mixer outlet 162 to the mixer inlets in the second subset.

This switching between different subsets of the mixer inlets 144 can continue to move in a clockwise direction to different subsets that are each less than all of the mixer inlets 144. The subsets of mixer inlets 144 can be arranged differently than discussed above, where each subset included two mixer inlets 144 on one side of the fluid reservoir 110 and one mixer inlet on an opposite side of the fluid reservoir 110. For example, a subset may include only a single mixer inlet 144, only a pair of mixer inlets 144 that are adjacent to each other, only a pair of mixer inlets 144 that are on opposite sides of the fluid reservoir 110 from each other, etc. Also, during some periods of time, no mixing may occur (in which case, the mixer pump 160 can be deactivated). And during some periods of time, all the mixer inlets 144 may be active, with recirculating fluid 158 flowing from the stored fluid 132 through all of the mixer inlets 144 during the same period of time.

In alternative examples, the fluid reservoir system 100 can include different numbers of mixer inlets 144 and/or mixer outlets 162. Also, the mixer inlets 144 and/or the mixer outlets 162 can be structured differently in different examples. For example, the mixer inlets may be holes in one or more pipes that extend around the periphery of the fluid reservoir 110 and are at least partially submerged in the stored fluid 132.

The fluid reservoir system 100 can also include a sediment removal system 210. The sediment removal system 210 can include a sediment removal inlet 212 (or possibly multiple sediment removal inlets). For example, as with the mixer outlet 162, the single sediment removal inlet 212 can extend down through the concrete of the bottom drain area 122 and to a sediment removal conduit 214, which can extend up to a single sediment removal outlet 215 to the sediment removal reservoir 216, which can be an in-ground sump pit such as a concrete tank. The sediment removal conduit 214 can include one or more valves in the sediment removal conduit 214, such as an operating valve 218 that can be used for normal control of flow through the sediment removal conduit 214, and a service valve 220 that can be closed for servicing (such as for servicing the operating valve 218, or for servicing other features in the sediment removal reservoir 216). The sediment removal system 210 may include a conduit structure 219 with just a single sediment removal conduit 214 in designs that use a single sediment sump such as the single bottom drain area 122 as illustrated in FIGS. 1-10 or with multiple conduits in the liner V design that can use multiple sediment evacuation sumps 222 in a bottom drain area 122 for multiple sediment removal conduits 214 as illustrated in FIG. 11. Each of these conduits 214 can have a single inlet like the sediment removal inlet 212 from the fluid reservoir 110 and a single outlet like the sediment removal outlet 215 to the sediment removal reservoir 216. Each of these conduits 214 may also include other components such as the operating valve 218 and the service valve 220. A sediment removal pump 230 can be a submersible pump that is positioned in the sediment removal reservoir 216, so that the sediment removal pump 230 can pump fluid out of the sediment removal reservoir 216 and to another fluid storage location such as a reservoir that is operating as a post digester storage (not shown).

The sediment removal reservoir 216 can be located so that the bottom of the sediment removal reservoir 216 is below a normal operating surface of the stored fluid 132 in the fluid reservoir 110. Thus, the sediment removal pump 230 can pump fluid from the sediment removal reservoir 216 until a top surface 232 of sediment removal fluid 233 (see FIG. 11) in the sediment removal reservoir 216 is below (i.e., at a lower altitude than) the top surface 146 of the stored fluid 132 in the fluid reservoir 110 by an altitude difference. The sediment removal fluid 233 can be pumped from the sediment removal reservoir 216 until that altitude difference is sufficient to create a pressure head between the fluid reservoir 110 and the sediment removal reservoir 216 to force sediment and associated fluid through the sediment removal inlet 212, through the sediment removal conduit 214, and into the sediment removal reservoir 216. For example, this pressure head may be created by a sufficiently large altitude difference between the top surface 146 of the stored fluid 132 in the fluid reservoir 110 and the top surface 232 of sediment removal fluid 233 in the sediment removal reservoir 216. The sediment removal fluid 233 (which includes the sediment removed from the bottom of the fluid reservoir 110) can be pumped out of the sediment removal reservoir 216 by the sediment removal pump 230. That exiting fluid can then travel through a conduit to another storage location such as a post digester storage.

The mixer 140 can operate during or prior to removal of sediment using the sediment removal system 210. For example, this may inject low solid fluid near the sediment removal inlet 212, which can facilitate greater flow of the fluid that includes the sediment to be removed.

The fluid reservoir system 100 can also include a leak detection monitoring pipe. 240. For example, this leak detection monitoring pipe 240 can be a pipe that extends under the fluid reservoir 110. Periodically, liquid can be pumped from the leak detection monitoring pipe 240 using a pump 242 (see FIG. 11), and it can be determined whether the fluids pumped from the leak detection monitoring pipe 240 contain biological material from the fluid reservoir 110 (or from one of the conduits), which would indicate that fluid is leaking from the fluid reservoir system 100. Further work can then be done to locate and fix the leak.

Referring to FIG. 11, the fluid reservoir 110 can include a cover 250, which can be a bio membrane that spans the top of the fluid reservoir 110, defining a contained space above the stored fluid 132 where biogas emitted from the stored fluid 132 can accumulate. The edges of the cover 250 can sealed, such as by being clamped, welded, or buried in the ground 114 outside of the mixer inlets 144. The conduits and pipes discussed herein can pass beneath the edges of the cover as illustrated in schematic form in FIG. 11, so that the cover 250 need not include holes to accept the conduits or have the associated stress points on the cover 250.

The fluid reservoir system 100 can also include a gas collection system 260. The gas collection system 260 can include gas collection inlets 262 that can extend through the concrete mixer inlets 144. However, the gas collection inlets 262 can be positioned above fluid levels in the mixer inlets 144 during normal operation of the fluid reservoir system 100. The gas collection inlets 262 can be fluidly connected to gas collection conduits 264, which can lead to other gas collection components to capture and store biogas that passes through the gas collection inlets 262 and gas collection conduits 264 as the biogas is emitted from the stored fluid 132 and contained by the fluid reservoir 110. The gas collection conduits 264 can pass beneath the edges of the cover 250 so that the gas collection conduits need not be supported on top of the cover 250 and/or extend through holes in the cover 250 that could create stress points and possible sources of leaks from the cover 250.

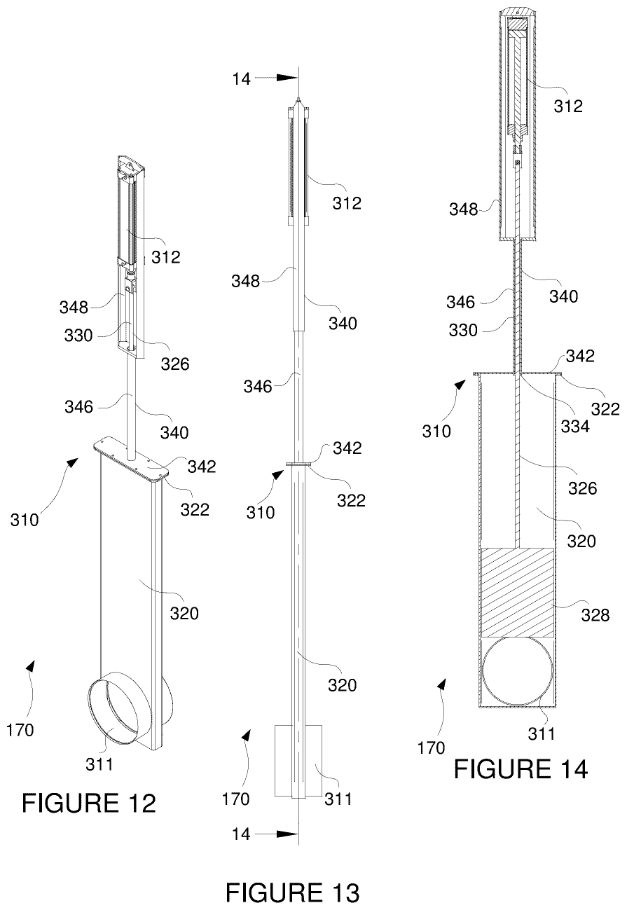

Referring to FIGS. 12-17, an example of a mixer inlet valve 170 will be discussed in more detail. This example mixer inlet valve 170 can be designed so that the disassembly service connection 310 is at a higher elevation than the top surface 146 of the stored fluid 132 of the fluid reservoir 110, even though inlet conduit 154 passing through the mixer inlet valve 170 at the valve's conduit portion 311 can be below the altitude of the top surface 146 of the stored fluid 132 of the fluid reservoir 110. Accordingly, for example, if someone hit the valve actuator 312 that is exposed above the ground with a vehicle and damaged the valve actuator 312, the mixer inlet valve 170 could be disassembled and damaged parts replaced, or the shaft seal replaced while the fluid reservoir system 100 is still full and in operation.

More specifically, the example mixer inlet valve 170 can include an outer lower housing 320, which may be made from cut and bent stainless steel and short pieces of stainless-steel pipe welded together. The outer lower housing 320 can include the conduit portion 311 and can extend up to a lower service connection flange 322 at the disassembly service connection 310, which can be at the top of the outer lower housing 320. The mixer inlet valve 170 can also include an inner moving knife valve portion 326, which can be made from a flat gate 328 of stainless-steel plate and a smooth round shaft 330 attached to the plate with a travel stop 334 attached to the shaft 330. The flat gate 328 and at least part of the shaft 330 can move within the lower housing 320, with the shaft 330 extending above the lower housing 320 and through an upper housing 340. The upper housing 340 can include an upper service connection flange 342, which can be bolted to the lower service connection flange 322. The upper housing 340 can also include a tube 346 extending up from the lower service connection flange 322, with shaft 330 extending through the tube 346. A top of the tube 346 can be connected to an actuator frame 348, which can be connected to the valve actuator 312. The valve actuator 312 can be connected to the shaft 330 so that the valve actuator can move the inner moving knife valve portion 326. For example, the valve actuator 312 may include a powered gas ram such as an air ram, which can be controlled by the controller 550. As an alternative, the valve actuator 312 could be manual actuator such as a manually operated screw actuator.

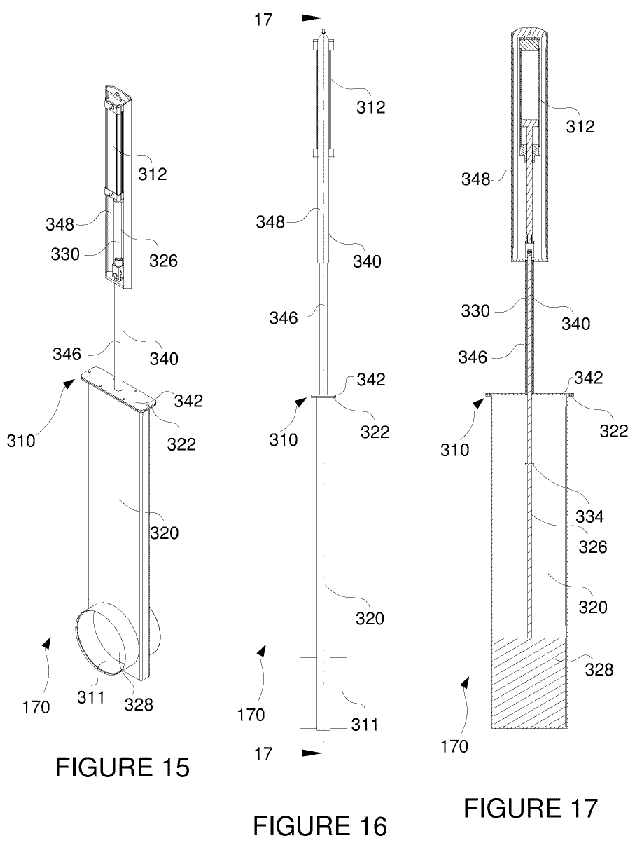

The valve actuator 312 can move the knife valve portion 326 from the open position illustrated in FIGS. 12-14 where the gate 328 does not block the flow of fluid through conduit portion 311 of the valve to the closed position illustrated in FIGS. 15-17 where the gate 328 does block the flow of fluid through the conduit portion 311 of the valve. Alternatively, a different valve such as a standard valve can be used as a mixer inlet valve, but the example mixer inlet valve 170 may reduce valve maintenance hassle and expenses.

### III. Second Example Fluid Reservoir System

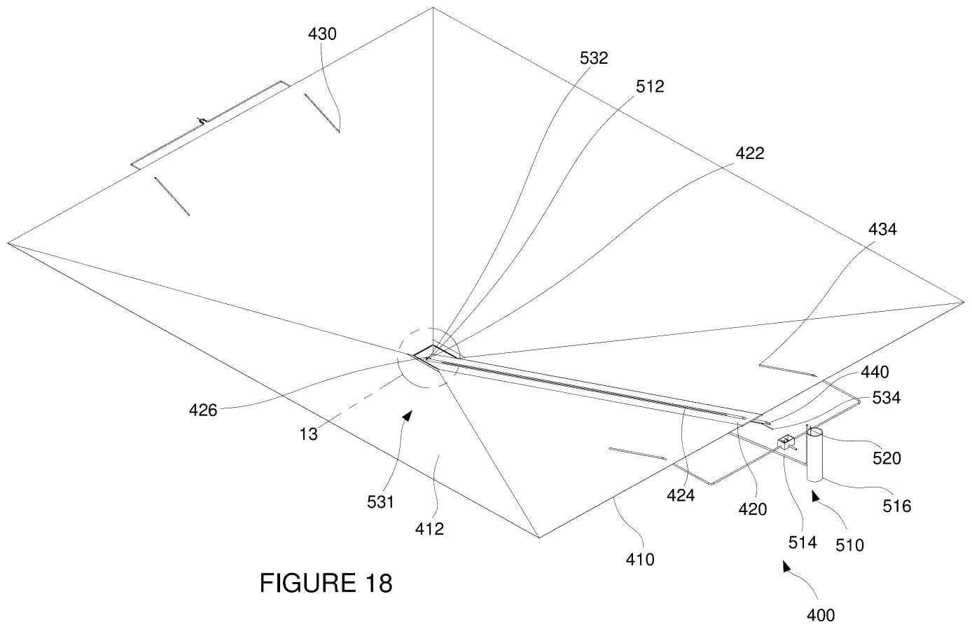

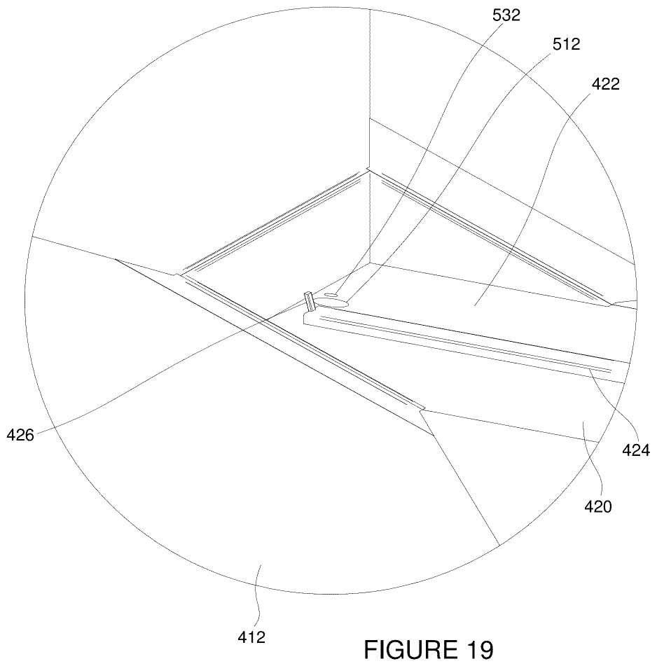

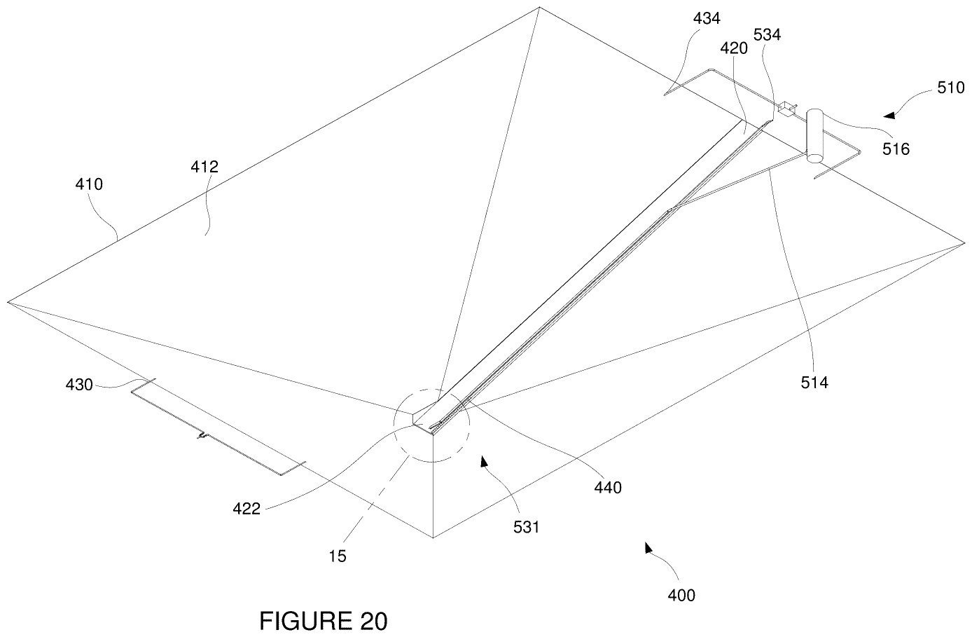

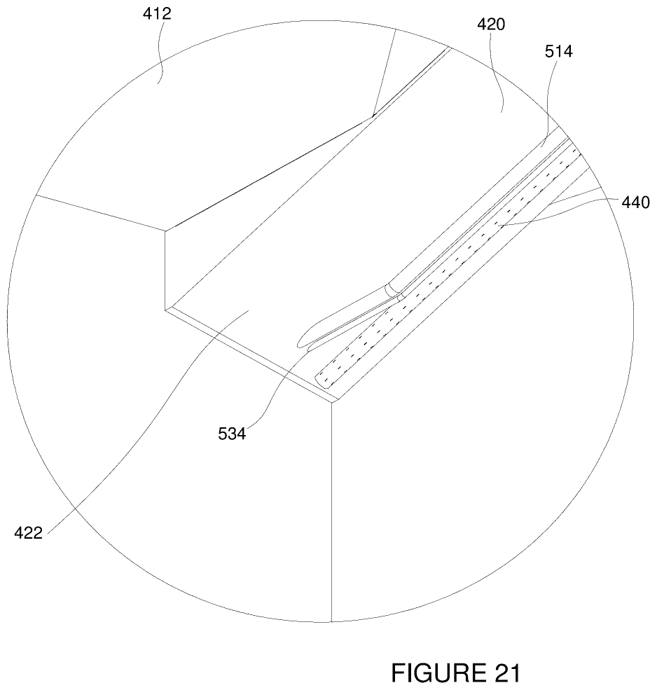

Referring now to FIGS. 18-21, another example of a fluid reservoir system 400 will be discussed. The fluid reservoir system 400 can be like the fluid reservoir system 100, except that the fluid reservoir system 400 can omit the mixer. For example, the fluid reservoir system 400 can include a fluid reservoir 410, a liner 412, a service ramp 420, a bottom drain area 422, a guide rail 424 and stop 426 on the service ramp 420, a pair of fluid inlets 430, and a pair of overflow outlets 434. The fluid reservoir system 400 can also include a sediment removal system 510, which can include a sediment removal inlet 512, a sediment removal conduit 514, a sediment removal reservoir 516, an operating valve (not shown), a service valve 520, and a sediment removal pump (not shown).

The sediment removal system 510 can also include a fluid injector 531 having a fluid injection outlet 532 at the bottom drain area 422 near the sediment removal inlet 512. The fluid injector 531 can also include a fluid injection conduit 534 connected to the fluid injection outlet 532. The fluid injection conduit 534 can be connected to a fluid injection pump (not shown) to pump low solids fluid through the fluid injection conduit 534 to the fluid injection outlet 532. The fluid injection pump can receive low solids fluid from a source such as fluid from the top of the fluid reservoir 410 or some other source. The low solids fluid can be pumped through the fluid injection outlet 532 into the fluid reservoir 410 near the sediment removal inlet 512 during or prior to flowing sediment-containing fluid into the sediment removal inlet 512 to aid the flow into the sediment removal inlet 512. As discussed above, the mixer 140 can provide similar assistance to the sediment removal system 210 in the fluid reservoir system 100.

The fluid reservoir system 400 can also include a leak detection monitoring pipe 440 like the leak detection monitoring pipe 240 of the fluid reservoir system 100. Also, the fluid reservoir system 400 can include a cover (not shown) on the fluid reservoir 410 to capture biogas from the fluid in the fluid reservoir 410. Gas collection inlets and gas collection conduits can also be included in the fluid reservoir system 400. For example, the gas collection conduits may pass through holes in the cover, through holes in the liner, or between the cover and liner so that the gas collection inlets at the ends of the gas collection conduits are fluidly connected to the space between the top surface of fluid in the fluid reservoir 110 and the cover.

The conduit pipes, pumps, and valves for the fluid reservoir system 100 and the fluid reservoir system 400 can be standard types of such components, although in some situations custom components may be used, such as for the mixer inlet valves 170, as discussed above with reference to FIGS. 12-17. For example, the pipes may be polymer pipes such as polyvinyl chloride (PVC) or high-density polyethylene (HDPE) pipes. Larger pipes may be chosen to reduce mixing energy required, such as one-foot diameter pipes for the mixer inlet conduits, two-foot diameter pipes for the main pipes extending around the fluid reservoir 110 and down to the mixer outlet of the mixer system, about eight-inch to ten-inch pipes for the reservoir inlets and overflow outlets as well as the leak detection pipes and the sediment removal conduit pipes. However, these are just examples, and other types and sizes of pipes may be used. Also, the pumps may be various types of pumps, such as axial flow pumps, mixed flow pumps, and/or other types of pumps. And the valves can be various types, such as gate valves, butterfly valves, and/or other types of valves.

Valves and pumps in the fluid reservoir system 100 and the fluid reservoir system 400 may be valves and pumps that can be controlled by a controller 550 (such as for the valves to be actuated between open and closed positions and the pumps to be activated and deactivated in response to control signals from the controller 550), such as a computerized controller illustrated in FIG. 11. Such a controller can be programmed to control the timing for activating and deactivating pumps, and for opening and closing valves. The timing of doing so can be determined by skilled operators of the fluid reservoir. And adjustments may be made after analyzing the results of different timing configurations, and other configurations such as determining which mixer inlets to include together in subsets for activation during different time periods. Such operators can also determine how often and for what duration different operations should be performed, such as mixing, sediment removal, flowing new fluid into the fluid reservoir inlets, and leak detection. Such timing and configuration parameters can be programmed into the controller 550. Alternatively, some or all of the activations and actuations of pumps and valves may be performed manually and/or with non-computerized timers.

Construction of the fluid reservoir system can use standard construction techniques and some techniques that are particular to the features discussed herein, such as techniques for ground preparation and installing concrete, bio membranes, pipes, valves, pumps, and controllers. As noted, the edges of the cover can be sealed in place.

### IV. Controller Computing Environment

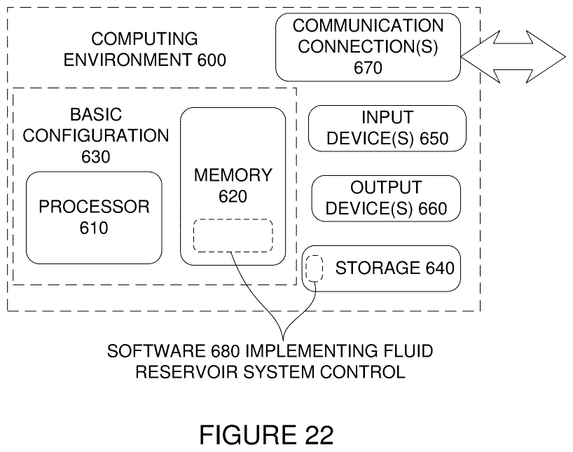

FIG. 22 illustrates a generalized example of a suitable computing environment 600 in which one or more aspects of the controller 550 may be implemented. For example, one or more such computing environments can be used as a controller, such as the controller 550. Generally, various different computing system configurations can be used. Examples of well-known computing system configurations that may be suitable for use with the tools and techniques described herein include, but are not limited to, server farms and server clusters, personal computers, server computers, smart phones, laptop devices, slate devices, game consoles, multiprocessor systems, microprocessor-based systems, programmable consumer electronics, network PCs, minicomputers, mainframe computers, distributed computing environments that include any of the above systems or devices, and the like.

The computing environment 600 is not intended to suggest any limitation as to scope of use or functionality of the invention, as the present invention may be implemented in diverse types of computing environments.

With reference to FIG. 22, various illustrated hardware-based computer components will be discussed. As will be discussed, these hardware components may store and/or execute software. The computing environment 600 includes at least one processing unit or processor 610 and memory 620. In FIG. 22, this most basic configuration 630 is included within a dashed line. The processing unit 610 executes computer-executable instructions and may be a real or a virtual processor. In a multi-processing system, multiple processing units execute computer-executable instructions to increase processing power. The memory 620 may be volatile memory (e.g., registers, cache, RAM), non-volatile memory (e.g., ROM, EEPROM, flash memory), or some combination of the two. The memory 620 stores software 680 implementing fluid reservoir system control, such as being configured to control a fluid reservoir system, which may include receiving sensor data from components of the fluid reservoir system and sending wired and/or wireless signals to control components of the fluid reservoir system such as valves and pumps in the system. An implementation of fluid reservoir control may involve all or part of the activities of the processor 610 and memory 620 being embodied in hardware logic as an alternative to or in addition to the software 680.

Although the various blocks of FIG. 22 are shown with lines for the sake of clarity, in reality, delineating various components is not so clear and, metaphorically, the lines of FIG. 22 and would more accurately be grey and blurred. For example, one may consider a presentation component such as a display device to be an I/O component (e.g., if the display device includes a touch screen). Also, processors have memory. The inventors hereof recognize that such is the nature of the art and reiterate that the diagram of FIG. 22 is merely illustrative of an exemplary computing device that can be used in connection with one or more aspects of the technology discussed herein. Distinction is not made between such categories as “workstation,” “server,” “laptop,” “handheld device,” etc., as all are contemplated within the scope of FIG. 1 and reference to “computer,” “computing environment,” or “computing device.”A computing environment 600 may have additional features. In FIG. 22, the computing environment 600 includes storage 640, one or more input devices 650, one or more output devices 660, and one or more communication connections 670. An interconnection mechanism (not shown) such as a bus, controller, or network interconnects the components of the computing environment 600. Typically, operating system software (not shown) provides an operating environment for other software executing in the computing environment 600, and coordinates activities of the components of the computing environment 600.

The memory 620 can include storage 640 (though they are depicted separately in FIG. 22 for convenience), which may be removable or non-removable, and may include computer-readable storage media such as flash drives, magnetic disks, magnetic tapes or cassettes, CD-ROMs, CD-RWs, DVDs, which can be used to store information and which can be accessed within the computing environment 600. The storage 640 stores instructions for the software 680.

The input device(s) 650 may be one or more of various different input devices. For example, the input device(s) 650 may include a user device such as a mouse, keyboard, trackball, etc. The input device(s) 650 may implement one or more natural user interface techniques, such as speech recognition, touch and stylus recognition, recognition of gestures in contact with the input device(s) 650 and adjacent to the input device(s) 650, recognition of air gestures, head and eye tracking, voice and speech recognition, sensing user brain activity (e.g., using EEG and related methods), and machine intelligence (e.g., using machine intelligence to understand user intentions and goals). As other examples, the input device(s) 650 may include a sensor such as a gas and/or fluid flow sensor; a fluid level sensor; a temperature sensor; a scanning device; a network adapter; a CD/DVD reader; or another device that provides input to the computing environment 600. The output device(s) 660 may be a display, printer, speaker, CD/DVD-writer, network adapter, or another device that provides output from the computing environment 600. The input device(s) 650 and output device(s) 660 may be incorporated in a single system or device, such as a touch screen or a virtual reality system.

The communication connection(s) 670 enable communication over a communication medium to another computing entity, including to a controlled component such as a controlled valve or pump. Additionally, functionality of the components of the computing environment 600 may be implemented in a single computing machine or in multiple computing machines that are able to communicate over communication connections. Thus, the computing environment 600 may operate in a networked environment using logical connections to one or more remote computing devices, such as a handheld computing device, a personal computer, a server, a router, a network PC, a peer device, or another common network node. The communication medium conveys information such as data or computer-executable instructions or requests in a modulated data signal. A modulated data signal is a signal that has one or more of its characteristics set or changed in such a manner as to encode information in the signal. By way of example, and not limitation, communication media include wired or wireless techniques implemented with an electrical, optical, RF, infrared, acoustic, or other carrier.

The tools and techniques can be described in the general context of computer-readable media, which may be storage media or communication media. Computer-readable storage media are any available storage media that can be accessed within a computing environment, but the term computer-readable storage media does not refer to propagated signals per se. By way of example, and not limitation, with the computing environment 600, computer-readable storage media include memory 620, storage 640, and combinations of the above.

The tools and techniques can be described in the general context of computer-executable instructions, such as those included in program modules, being executed in a computing environment on a target real or virtual processor. Generally, program modules include routines, programs, libraries, objects, classes, components, data structures, etc. that perform particular tasks or implement particular abstract data types. The functionality of the program modules may be combined or split between program modules as desired in various aspects. Computer-executable instructions for program modules may be executed within a local or distributed computing environment. In a distributed computing environment, program modules may be located in both local and remote computer storage media.

The subject matter defined in the appended claims is not necessarily limited to overcoming problems discussed herein or otherwise providing the benefits described herein. A particular implementation of the invention may provide all, some, or none of the benefits described herein. Although operations for the various techniques are described herein in a particular, sequential order for the sake of presentation, it should be understood that this manner of description encompasses rearrangements in the order of operations, unless a particular ordering is required. For example, operations described sequentially may in some cases be rearranged or performed concurrently. Techniques described herein with reference to flowcharts may be used with one or more of the systems described herein and/or with one or more other systems. Moreover, for the sake of simplicity, flowcharts may not show the various ways in which particular techniques can be used in conjunction with other techniques.

### V. Aspects of the Disclosure

Some aspects of the disclosure will now be discussed. In these aspects, features of implementations of these aspects may be combined together with the general aspects and with each other in any combination not precluded by specific language or by the nature of the features and aspects.

Some aspects can involve techniques. Each of the acts in these techniques can be performed in a fluid reservoir system. The fluid reservoir system can be structured to perform the acts of the techniques, as discussed herein. The fluid reservoir system may include a controller computing environment. For example, each technique may be performed in a system including a computer system that includes at least one processor and memory including instructions stored thereon that when executed by at least one processor cause at least one processor to perform and/or signal other components of the reservoir fluid system to perform at least part of the technique (memory stores instructions (e.g., object code), and when processor(s) execute(s) those instructions, processor(s) perform(s) and/or signals other components to perform the technique). Similarly, one or more computer-readable memory may have computer-executable instructions embodied thereon that, when executed by at least one processor, cause at least one processor to perform at least a portion of a technique. The techniques discussed below may be performed and/or initiated at least in part by hardware logic. Features discussed in each of the techniques below may be combined with each other in any combination not precluded by the discussion herein, including combining features from a technique discussed with reference to one figure in a technique discussed with reference to a different figure. Also, a computer system may include means for performing each of the acts discussed in the context of these techniques (such as via other components of the fluid reservoir system), in different combinations.

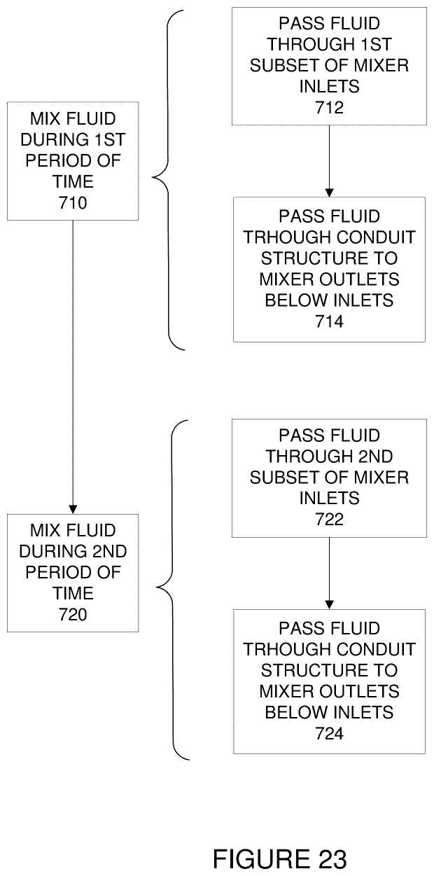

Referring to FIG. 23, according to one aspect, a technique can include mixing 710 fluid in a fluid reservoir during a first period of time with a first mixer system configuration. The mixing 710 can include passing 712 fluid from the fluid reservoir through a first subset of one or more mixer inlets that includes less than all of a set of mixer inlets positioned around the fluid reservoir. The mixing 710 can also include passing 714 fluid through a mixer conduit structure from the first subset of one or more mixer inlets and back into the fluid reservoir through at least one of one or more mixer outlets below the set of mixer inlets. Also, the technique can include mixing 720 fluid in the fluid reservoir during a second period of time that is different from the first period of time with a second mixer system configuration that is different from the first mixer system configuration. The technique may include reconfiguring from the first mixer system configuration to the second mixer system configuration, and this reconfiguring may include a controller sending signals to one or more system components. The mixing 720 can include passing 722 fluid from the fluid reservoir through a second subset of one or more mixer inlets including less than all of the set of mixer inlets, with the second subset of one or more mixer inlets being different from the first subset of one or more mixer inlets. The mixing 720 can also include passing 724 fluid through the mixer conduit structure from the second subset of one or more mixer inlets and back into the fluid reservoir through at least one of the one or more mixer outlets below the set of mixer inlets.

The first subset of one or more mixer inlets can include at least one mixer inlet that is also in the second subset of one or more mixer inlets. In other examples, the first subset of one or more mixer inlets may not include any mixer inlets that are also in the second subset of one or more mixer inlets. Also, the first subset of one or more mixer inlets and the second subset of one or more mixer inlets can each include at least one mixer inlet that is not in the other subset of one or more mixer inlets.

The mixer inlets can be at the top of the fluid in the fluid reservoir or within the top twenty-five percent of the height of the fluid in the fluid reservoir. The mixer outlets can be at the bottom of the fluid in the fluid reservoir or within the bottom twenty-five percent of the height of the fluid in the fluid reservoir.

The technique can further include switching from the first mixer system configuration to the second mixer system configuration, with the switching including closing one or more valves and opening one or more other valves. This switching may include a controller sending one or more signals to open and/or close one or more valves.

The technique can further include skimming material from a top surface of fluid in the fluid reservoir through at least one of the mixer inlets, wherein the set of mixer inlets includes at least one skimmer (such as the mixer inlets 144 discussed above) that is structured to skim solids (such as material from a crust and/or other solids) from a top surface of fluid in the reservoir.

The mixing 710 fluid in the fluid reservoir during the first period of time can include creating an upward flow of fluid in the reservoir from at least one of the one or more mixer outlets to the first subset of one or more mixer inlets. Also, the mixing 720 fluid in the fluid reservoir during the second period of time can include creating an upward flow of fluid in the reservoir from at least one of the one or more mixer outlets to the second subset of one or more mixer inlets.

The mixing 710 fluid in the fluid reservoir during the first period of time and mixing 720 fluid in the fluid reservoir during the second period of time can help to keep at least one of solid material and semisolid material suspended in the fluid reservoir.

A pattern of flow in the fluid reservoir during the first period of time can be different from a pattern of flow in the fluid reservoir during the second period of time.

The technique can further include pumping fluid from a sediment removal reservoir, the pumping of fluid lowering a fluid surface of the sediment removal reservoir to a level that is at a lower altitude than a level of a fluid surface of the fluid reservoir so that there is a difference in altitude of the fluid surface of the fluid reservoir and the fluid surface of the sediment removal reservoir. Accumulated sediment can be cleared from a bottom of the fluid reservoir by flowing the sediment in fluid from the fluid reservoir to the sediment removal reservoir through a sediment removal conduit structure using a pressure head due to the difference in altitude of the fluid surface of the fluid reservoir and the fluid surface of the sediment removal reservoir.

The conduit structure can include one or more conduits, with each of the one or more conduits having only a single inlet from the fluid reservoir and only a single outlet to the sediment removal reservoir.

The technique can further include injecting fluid adjacent to an inlet of the sediment removal conduit structure at a low point in the fluid reservoir, and the injecting of fluid can facilitate the flow of the sediment in the fluid from the fluid reservoir through the conduit structure. For example, this injecting may be done using the mixing techniques discussed above or with a separate injector, such as the fluid injector 531 discussed above.

The fluid reservoir can include a cover that captures biogas in an enclosed area above a surface of fluid in the fluid reservoir, wherein the fluid reservoir further includes at least one biogas collection conduit extending from the enclosed area and below the cover to pass captured biogas away from the fluid reservoir. For example, the biogas collection conduit may extend from a structure of an inlet of the set of mixer inlets.

According to another aspect, a fluid reservoir system can include a fluid reservoir and a mixer. The mixer can include multiple mixer inlets positioned around an upper perimeter of the fluid reservoir. A conduit structure can extend from the mixer inlets to one or more mixer outlets below the mixer inlets. A pump system can be fluidly connected to the conduit structure. The mixer can be structured to use the pump system to recirculate fluid to the reservoir by pumping fluid from the top of the reservoir through different subsets of the mixer inlets at different times, through the conduit structure, and through the one or more mixer outlets back into the fluid reservoir.

The mixer inlets can include skimmers that are structured to skim material from a surface of the fluid reservoir. The different subsets of the mixer inlets can include a first subset and a second subset, wherein the first subset can include at least one mixer inlet that is also in the second subset, and wherein the first subset can include at least one mixer inlet that is not in the second subset. The mixer can be structured such that operation of the mixer can help to keep at least one of solid material and semisolid material suspended in the fluid reservoir.

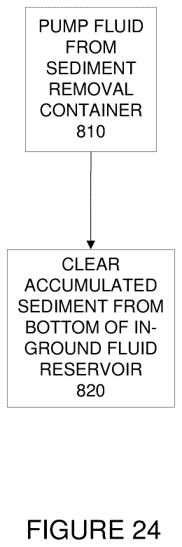

Referring to FIG. 24, according to another aspect, a technique can include pumping 810 fluid from a sediment removal reservoir. The pumping 810 can lower a fluid surface of the sediment removal reservoir to a level that is at a lower altitude than a level of a fluid surface of a fluid reservoir that extends at least partially into ground so that there is a difference in altitude of the fluid surface of the fluid reservoir and the fluid surface of the sediment removal reservoir. The technique can further include clearing 820 accumulated sediment from a bottom of the fluid reservoir by flowing the sediment in fluid from the fluid reservoir to the sediment removal reservoir through a conduit structure using a pressure head due to the difference in altitude of the fluid surface of the fluid reservoir and the fluid surface of the sediment removal reservoir.

The technique can further include injecting fluid adjacent to an inlet of the conduit structure at a low point in the fluid reservoir, the injecting of fluid can facilitate the flow of the sediment in the fluid from the fluid reservoir through the conduit structure. The injecting of fluid adjacent to the inlet of the conduit structure can be part of operating a recirculating mixer system for mixing fluid in the fluid reservoir.

The technique can further include passing the sediment from the sediment removal reservoir to a location that is different from the fluid reservoir.

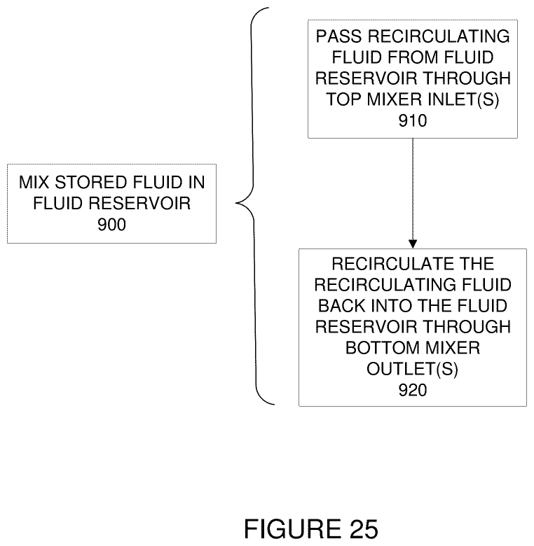

Referring to FIG. 25, according to yet another aspect, stored fluid comprising biological material in a fluid reservoir can be mixed 900. The mixing 900 can include passing 910 recirculating fluid from the fluid reservoir through one or more mixer inlets at a top of the stored fluid in the fluid reservoir, and recirculating 920 the recirculating fluid through a mixer conduit structure from the one or more mixer inlets and back into the fluid reservoir through at least one mixer outlet at a bottom of the stored fluid in the fluid reservoir.

The mixing 900 can produce an up flow in the stored fluid in the fluid reservoir between the one or more mixer outlets and the one or more mixer inlets. The up flow can produce an upward force (which may have horizontal components in addition to its upward force component) on solids in the stored fluid in the fluid reservoir.

While particular examples are discussed above, it will be understood by those skilled in the art that various changes in form and details may be made therein without departing from the spirit and scope of the disclosure.

I/We claim:

1. A method comprising:
mixing fluid comprising biological material in a fluid reservoir during a first period of time with a first mixer system configuration by performing the following:
passing fluid from the fluid reservoir through a first subset of one or more mixer inlets comprising less than all of a set of mixer inlets positioned around the fluid reservoir; and
passing fluid through a mixer conduit structure from the first subset of one or more mixer inlets and back into the fluid reservoir through at least one of one or more mixer outlets below the set of mixer inlets; and
mixing fluid in the fluid reservoir during a second period of time that is different from the first period of time with a second mixer system configuration that is different from the first mixer system configuration by performing the following:
passing fluid from the fluid reservoir through a second subset of one or more mixer inlets comprising less than all of the set of mixer inlets, the second subset of one or more mixer inlets being different from the first subset of one or more mixer inlets; and
passing fluid through the mixer conduit structure from the second subset of one or more mixer inlets and back into the fluid reservoir through at least one of the one or more mixer outlets below the set of mixer inlets.

2. The method of claim 1, wherein the first subset of one or more mixer inlets includes at least one mixer inlet that is also in the second subset of one or more mixer inlets.

3. The method of claim 1, wherein the first subset of one or more mixer inlets and the second subset of one or more mixer inlets each includes at least one mixer inlet that is not in the other subset of one or more mixer inlets.

4. The method of claim 1, further comprising switching from the first mixer system configuration to the second mixer system configuration, the switching comprising closing one or more valves and opening one or more other valves.

5. The method of claim 1, further comprising skimming material from a top surface of fluid in the fluid reservoir through at least one of the mixer inlets, wherein the set of mixer inlets comprises at least one skimmer that is structured to skim solids from a top surface of fluid in the fluid reservoir.

6. The method of claim 1, wherein the mixer inlets are at the top of the fluid in the fluid reservoir or within the top twenty-five percent of the height of the fluid in the fluid reservoir.

7. The method of claim 1, wherein the mixer outlets are at the bottom of the fluid in the fluid reservoir or within the bottom twenty-five percent of the height of the fluid in the fluid reservoir.

8. The method of claim 1, wherein mixing fluid in the fluid reservoir during the first period of time comprises creating an upward flow of fluid in the reservoir from at least one of the one or more mixer outlets to the first subset of one or more mixer inlets, and wherein mixing fluid in the fluid reservoir during the second period of time comprises creating an upward flow of fluid in the reservoir from at least one of the one or more mixer outlets to the second subset of one or more mixer inlets.

9. The method of claim 1, wherein mixing fluid in the fluid reservoir during the first period of time and mixing fluid in the fluid reservoir during the second period of time help to keep at least one of solid material and semisolid material suspended in the fluid reservoir.

10. The method of claim 1, wherein a pattern of flow in the fluid reservoir during the first period of time is different from a pattern of flow in the fluid reservoir during the second period of time, and wherein the pattern of flow during the first period of time focuses fluid flow in a different area than the pattern of flow during the second period of time.

11. The method of claim 1, further comprising:
pumping fluid from a sediment removal reservoir, the pumping of fluid lowering a fluid surface of the sediment removal reservoir to a level that is at a lower altitude than a level of a fluid surface of the fluid reservoir so that there is a difference in altitude of the fluid surface of the fluid reservoir and the fluid surface of the sediment removal reservoir;
clearing sediment from a bottom of the fluid reservoir by flowing the sediment in fluid from the fluid reservoir to the sediment removal reservoir through a sediment removal conduit structure using a pressure head due to the difference in altitude of the fluid surface of the fluid reservoir and the fluid surface of the sediment removal reservoir; and
injecting fluid adjacent to an inlet of the sediment removal conduit structure at a low point in the fluid reservoir, the injecting of fluid facilitating the flow of the sediment in the fluid from the fluid reservoir through the sediment removal conduit structure.

12. The method of claim 1, wherein the fluid reservoir comprises a cover that captures biogas in an enclosed area above a surface of fluid in the fluid reservoir, wherein the fluid reservoir further comprises at least one biogas collection conduit extending from the enclosed area and below the cover to pass captured biogas away from the fluid reservoir, and wherein the biogas collection conduit extends from a structure of an inlet of the set of mixer inlets.

13. A fluid reservoir system comprising:
a fluid reservoir; and
a mixer comprising:
multiple mixer inlets positioned around an upper perimeter of the fluid reservoir;
a conduit structure extending from the mixer inlets to one or more mixer outlets below the mixer inlets; and
a pump system fluidly connected to the conduit structure, the mixer being structured to use the pump system to recirculate fluid to the fluid reservoir by pumping fluid from the top of the fluid reservoir through different subsets of the mixer inlets at different times, through the conduit structure, and through the one or more mixer outlets back into the fluid reservoir.

14. The fluid reservoir system of claim 13, wherein the mixer inlets comprise skimmers that are structured to skim material from a surface of the fluid reservoir.

15. The fluid reservoir system of claim 13, wherein the different subsets of the mixer inlets include a first subset and a second subset, wherein the first subset includes at least one mixer inlet that is also in the second subset, and wherein the first subset includes at least one mixer inlet that is not in the second subset.

16. The fluid reservoir system of claim 13, wherein the mixer is structured such that operation of the mixer helps to keep at least one of solid fluid and semisolid fluid suspended in the fluid reservoir.

17. The fluid reservoir system of claim 13, wherein the mixer inlets are at the top of the fluid in the fluid reservoir or within the top twenty-five percent of the height of the fluid in the fluid reservoir.

18. The fluid reservoir system of claim 13, wherein the mixer outlets are at the bottom of the fluid in the fluid reservoir or within the bottom twenty-five percent of the height of the fluid in the fluid reservoir.

19. The fluid reservoir system of claim 13, further comprising a fluid removal system comprising:
a sediment removal reservoir;
a sediment removal conduit structure fluidly connecting the fluid reservoir to the sediment removal reservoir, the fluid reservoir being at least a partially in-ground reservoir; and
a pump that is structured to pump fluid from the sediment removal reservoir to lower a fluid surface of the sediment removal reservoir to a level that is at a lower altitude than a level of a fluid surface of the fluid reservoir so that there is a difference in altitude of the fluid surface of the fluid reservoir and the fluid surface of the sediment removal reservoir, the difference in altitude being structured to produce a head pressure to flow fluid comprising sediment from the fluid reservoir to the sediment removal reservoir through the sediment removal conduit structure.

20. A method comprising:
pumping fluid from a sediment removal reservoir, the pumping of fluid lowering a fluid surface of the sediment removal reservoir to a level that is at a lower altitude than a level of a fluid surface of a fluid reservoir that extends at least partially into ground so that there is a difference in altitude of the fluid surface of the fluid reservoir and the fluid surface of the sediment removal reservoir; and
clearing sediment from a bottom of the fluid reservoir by flowing the sediment in fluid from the fluid reservoir to the sediment removal reservoir through a conduit structure using a pressure head due to the difference in altitude of the fluid surface of the fluid reservoir and the fluid surface of the sediment removal reservoir.

21. The method of claim 20, wherein the conduit structure comprises one or more conduits, with each of the one or more conduits having only a single inlet from the fluid reservoir and only a single outlet to the sediment removal reservoir.

22. The method of claim 20, further comprising injecting fluid adjacent to an inlet of the conduit structure at a low point in the fluid reservoir, the injecting of fluid facilitating the flowing of the sediment in the fluid from the fluid reservoir through the conduit structure.

23. The method of claim 20, further comprising passing the sediment from the sediment removal reservoir to a location that is different from the fluid reservoir.

24. A method comprising:
mixing stored fluid comprising biological material in a fluid reservoir, the mixing comprising:
passing recirculating fluid from the fluid reservoir through one or more mixer inlets at a top of the stored fluid in the fluid reservoir; and
recirculating the recirculating fluid through a mixer conduit structure from the one or more mixer inlets and back into the fluid reservoir through at least one mixer outlet at a bottom of the stored fluid in the fluid reservoir.

25. The method of claim 24, wherein the mixing produces an up flow in the stored fluid in the fluid reservoir between the at least one mixer outlet and the one or more mixer inlets, the up flow producing an upward force on solids in the stored fluid in the fluid reservoir.
